(19) United States
(12) Patent Application Publication
(10) Pub. No.: US 2017/0216156 A1
(43) Pub. Date: Aug. 3, 2017

(54) PERFORMANCE-ENHANCED ANTIPERSPIRANTS

(71) Applicant: Henkel AG & Co. KGaA, Duesseldorf (DE)

(72) Inventors: Thomas Doering, Dormagen (DE); Jing Zhou, Shanghai (CN)

(73) Assignee: Henkel AG & Co. KGaA, Duesseldorf (DE)

(21) Appl. No.: 15/492,580

(22) Filed: Apr. 20, 2017

Related U.S. Application Data
(63) Continuation of application No. PCT/EP2015/071778, filed on Sep. 22, 2015.

(30) Foreign Application Priority Data
Oct. 23, 2014 (DE) 10 2014 221 531.5

(51) Int. Cl. A61K 8/02; A61K 8/86; A61K 8/28; A61K 8/37; A61K 8/34; A61K 8/26; A61Q 15/00; A61K 8/893

(52) U.S. Cl. CPC A61K 8/0229; A61Q 15/00; A61K 8/86; A61K 8/893; A61K 8/37; A61K 8/342; A61K 8/26; A61K 8/28

(57) ABSTRACT

The object of the present invention is an antiperspirant stick including (A) at least one alkoxy-modified silicone, (B) at least one ester of a C8-C14 alkanoic acid and a C8-C14 alkanol, and (C) at least one antiperspirant active ingredient from the group of aluminum salts or aluminum-zirconium salts.

### FIELD OF THE INVENTION

The present invention generally relates to cosmetic and dermatological antiperspirant sticks having improved antiperspirant efficacy and improved texture.

### BACKGROUND OF THE INVENTION

Compositions against body odor are an important part of daily personal hygiene. They should ensure that the sweat formed during the day through various activities (physical exercise, work, sports), but also formed by psychological strain does not lead to unpleasant body odor. The deodorant active ingredients of commercially available deodorants are just as varied as the components of the sweat and the causes of the development of body odor. Odor absorbers, fragrances, deodorizing ion exchangers, antimicrobial agents, prebiotically active components and enzyme inhibitors can be used as cosmetic deodorizing active ingredients. In a simplified illustration, body odor can be attributed to the bacterial decomposition of the organic constituents of sweat. Some of the bacteria typical for the natural microflora of the human skin bacteria are responsible for bacterial decomposition, in particular gram-positive anaerobic cocci, for example staphylococci, such as Staphylococcus hominis, and Coryne bacteria. Since the body odor is caused by bacterial activity, it can be prevented particularly effectively through the application of cosmetic products (soaps, creams, powders, sticks, rollers, gels or sprays) which include antimicrobially active ingredients and perfume oil compositions.

The aerosol spray, roll-on and antiperspirant sticks have become established in the market as application forms for said compositions. Furthermore, the deodorant in powder form (also as a compacted powder) or the deodorant applied to a disposable substrate (such as a cloth, pad or puff) are also known. As a particularly pleasant application form, a person skilled in the art is aware of what are known as antiperspirant sticks or cream sticks (“soft solids”). These are understood to mean viscous compositions which possess a creamy texture and which are pushed out prior to use through one or more openings of a dispensing device of an applicator.

Antiperspirant sticks are generally produced with a low water content or are anhydrous. The anhydrous delivery form is especially preferred compared to aqueous systems when a particularly high antiperspirant efficacy is desired, because particularly effective antiperspirant active ingredients such as activated aluminum chlorohydrate are not stable in the long term in aqueous media.

In the case of produced antiperspirants having a low water content, preferably anhydrous antiperspirants, the optimum release of the antiperspirant active ingredients at the site of action constitutes a very specific challenge. The wax matrix commonly used in antiperspirants of this type surrounds the aluminum and/or aluminum/zirconium salts acting in an antiperspirant manner. Their contact with the water phase is thus restricted or even prevented. In the case of application of the antiperspirant to the skin, the active ingredients might then dissolve in insufficient quantity in the water phase (sweat), whereby the antiperspirant efficacy of the agents is greatly impaired.

A person skilled in the art will therefore always attempt to increase the release of antiperspirant active ingredients and/or to increase the efficiency of the antiperspirant active ingredients. Amphiphilic (surfactant) substances have hitherto been used to improve the release. Through the use of these amphiphilic substances, the hydrophobicity of the wax matrix may be reduced and thus the active ingredient availability can be increased. The improvements achieved in this way, however, are effective only in the short term, as amphiphilic substances at the same time also promote the entry of water into the anhydrous composition. This in turn causes a hardening of the surface of the stick-shaped antiperspirant agent over time. This hardening results in a lack of product discharge at the site of action, resulting in deterioration of the antiperspirant effect. In addition, a surface encrusted by the infiltration of water also greatly impairs further cosmetic product properties. The antiperspirant agent loses its creamy texture, and hardened areas located at the surface of the agent can cause discomfort for the user.

To increase the efficacy of antiperspirant products, the contents of aluminum salts are often significantly increased. This practice is not only uneconomical, but in addition can lead to skin incompatibilities.

The object of the present invention was therefore to provide a cosmetic composition having a low water content, preferably an anhydrous cosmetic composition for combating body odor, which cosmetic composition has an improved efficacy of the antiperspirant active ingredients. The release of the antiperspirant active ingredients at the site of action should also be increased. At the same time, the application properties of the agent in terms of its texture (for example its creamy consistency and the ability of the agent to glide over the skin after repeated application) should also be improved, and in this way a uniform product delivery will be ensured.

### BRIEF SUMMARY OF THE INVENTION

The subject of the present invention in a first embodiment is in antiperspirant stick, including
a) at least one alkoxy-modified silicone,
b) at least one ester of a C₈-C₁₄ alkanoic acid and a C₈-C₁₄ alkanol, and
c) at least one antiperspirant active ingredient from the group of aluminum salts or aluminum-zirconium salts.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it has now been found that this object can be solved in an outstanding manner if an antiperspirant active ingredient is used in antiperspirant sticks in combination with at least one alkoxy-modified silicone and at least one ester of a C8-C14 alkanoic acid and a C8-C14 alkanol.

Within the scope of this invention, high-viscosity compositions produced with a low water content also is to be anhydrous are understood as antiperspirant sticks. This composition can be a dispersion of fine-particle astringent aluminum or zirconium salts in a non-polar carrier liquid solidified by gelling agents or waxy fat components. For example, hydrocarbons or silicone oils, preferably those having boiling points below 200° C. or with a certain volatility, for example cyclic siloxanes (cyclomethicones) and mixtures thereof with skin-softening oil components are used as non-polar carrier liquids. The composition is usually cast or pressed in a stick shape, cylinder shape or conical shape and is offered packaged in an appropriate wrapping, from which it can be unscrewed or pressed just before use. The antiperspirant sticks according to the invention which include the essential ingredients (A), (B) and (C) are understood to mean compositions (preferably cast or molded in a stick shape, cylinder shape or conical shape) without further packaging or wrapping material.

As the first ingredient (A) essential to the invention, the antiperspirant sticks of the present invention include at least one alkoxy-modified silicone. Silicones are polymeric compounds which are constructed in accordance with the schema (R2SiOx), wherein R usually stands for an organic group, often an alkyl group also a substituted alkyl group. Silicones are also referred to as polyorganosiloxanes, and can be linear or branched. The silicon atoms are linked via oxygen atoms in a Si—O—Si bond.

Alkoxy-modified silicones are understood to mean silicones which carry at least one alkoxy group. Alkoxy groups are understood to be C₂-C₁₀ alkoxy groups. The alkoxy group can be located terminally on the silicone (i.e. for example present as a group —O—CH₃ or as a group —O—CH₂—CH₃). However, it is also possible in accordance with the invention if the alkoxy group itself also carries a substituent; in this case, an alkoxy modification is understood to mean a grouping located on the silicone, for example (—CH2-CH2-O—), (—CH2-CH2-CH2-O—), (—CH(CH3)-CH2-O—), (—CH2-CH(CH3)-CH2-O—) or (—CH2-CH2-CH2-CH2-O—). The alkoxy-modified silicones (A) according to the invention preferably carry at least one grouping (—CH2-CH2-O—) and/or (—CH2-CH2-CH2-O—).

The alkoxy groups can be linked to the silicone either via a carbon atom or via an oxygen atom, by way of example the silicones can carry the structural units of formula (a), (b), (c) and/or (d):

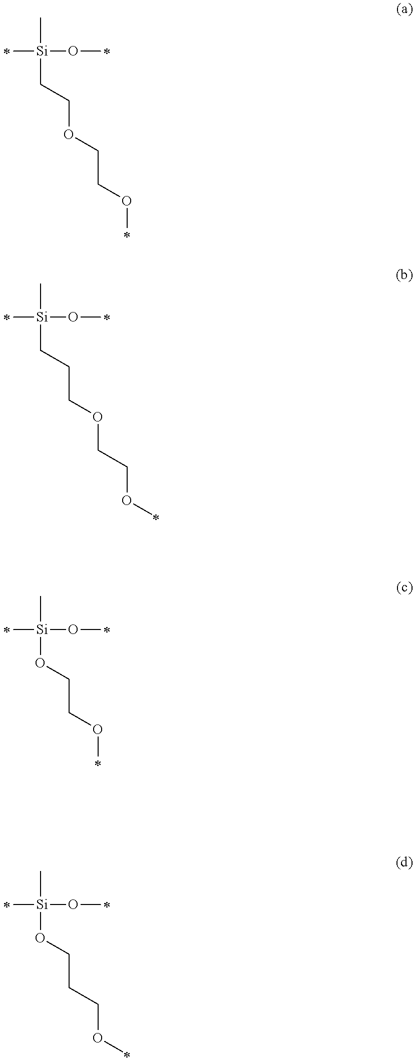

It is particularly preferred if the alkoxy-modified silicone(s) (A) carries/carry more than one alkoxy group, i.e. if the silicones (A) are polyalkoxylated. Polyalkoxylated silicones carry, as structural units, polyoxyalkylene groups, in particular polyoxyethylene groups (i.e. groups of the type [—CH2-CH2-O—]ₘ and/or polyoxypropylene groups (i.e. groups of the type [—CH(CH3)-CH2-O—]ₘ and/or [—CH2-CH2-CH2-O—]ₘ). The number of polyoxyalkylene units in the silicone polymer is preferably at least 2.

The alkoxy-modified silicone (A) is particularly preferably a non-ionic silicone. Non-ionic silicones carry neither positive nor negative charges.

In a further embodiment, what is preferred is in antiperspirant stick including
a) at least one non-ionic alkoxy-modified silicone,
b) at least one ester of a C₈-C₁₄ alkanoic acid and a C₈-C₁₄ alkanol, and
c) at least one antiperspirant active ingredient from the group of aluminum salts or aluminum-zirconium salts.

Alkoxy-modified silicones (A) which have proven to be particularly preferred are those which have at least one structural unit of formula (A1) presented below

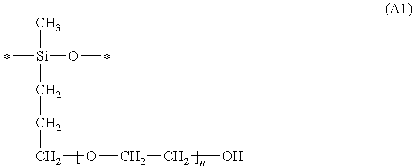

in which n stands for an integer from 2 to 20, preferably for an integer from 4 to 18, more preferably for an integer from 6 to 16, even more preferably for an integer from 8 to 14, and very particularly preferably stands for the number 12.

If silicones (A) having at least one structural unit (A1) were used in the antiperspirant sticks according to the invention, a particularly effective inhibition of the formation of sweat could be observed. At the same time, these antiperspirant sticks are characterized by a particularly creamy structure.

In a preferred embodiment, and antiperspirants stick according to the invention is therefore characterized in that it includes an alkoxy-modified silicone (Aa) which has at least one structural unit of formula (A1),

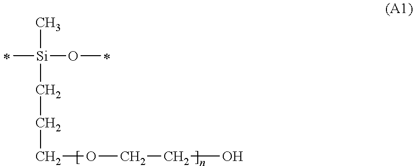

in which n stands for an integer from 2 to 20, preferably for an integer from 4 to 18, more preferably for an integer from 6 to 16, even more preferably for an integer from 8 to 14, and very particularly preferably stands for the number 12.

A preferred silicone (A) according to the invention can include, besides one or more structural units of general formula (A1), also further structural units which differ structurally from the units of formula (A1). The silicone (A) particularly preferably additionally comprises one or more dimethylsiloxane units. Depending on whether the silicone according to the invention is linear or branched, it has two (in the case of a chain-like, linear silicone) or more (in the case of a branched silicone) terminal groups. It has proven to be particularly advantageous if a silicone (A) according to the invention in each case has a trimethylsilyloxy group (i.e. a group —O—Si(CH₃)₃) as terminal group.

In a further very particularly preferred embodiment, an antiperspirant stick according to the invention is therefore characterized in that it includes an alkoxy-modified silicone (A) which is composed of structural units of formula (A1), of formula (A2), of formula (A3), and of formula (A3′),

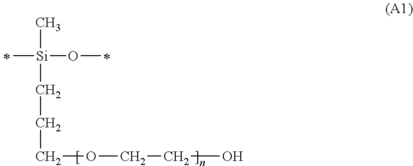

in which n (independently in each structural unit (A1)) stands in each case for an integer from 2 to 20, preferably for an integer from 4 to 18, more preferably for an integer from 6 to 16, even more preferably for an integer from 8 to 14, and very particularly preferably stands for the number 12,

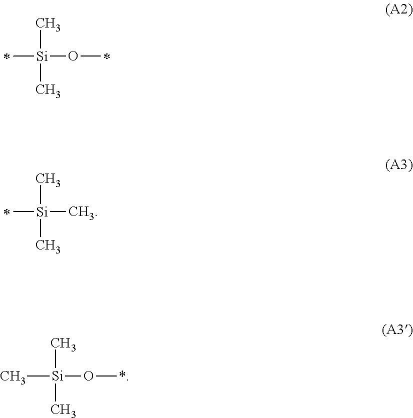

A silicone (A) which is composed of structural units of formula (A1), of formula (A2), of formula (A3), and of formula (A3′) is understood in this context to mean a silicone which has exclusively (in each case one or more) structural units of formulas (A1), (A2), (A3) and (A3′). Here, the silicone can also include various structural units of formula (A1), which differ in each case by their number n.

The positions characterized in the above structural units by a star in each case represent the linking points to the other structural units. By way of example, a very particularly preferred silicone composed of structural units of formula (A1), of formula (A2), of formula (A3) and of formula (A3′) can have the following structure:

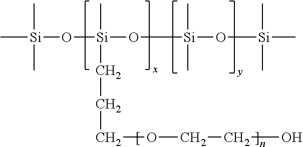

x and y are selected here depending on the desired molecular weight of the silicone, and n represents one of the above-described preferred or particularly preferred integers according to the invention.

Alkoxy-modified silicones both of low molecular weight and high molecular weight can be used as silicones (A). Particularly advantageous effects have been observed with silicones (A) having a molar mass of from 800 to 10,000 g/mol, preferably from 1,000 to 9,000 g/mol, more preferably from 2,000 to 8,000 g/mol, and particularly preferably from 2,500 to 5,000 g/mol.

If silicones (A) having a molar mass of from 2,500 to 5,000 g/mol have been used in the antiperspirant sticks according to the invention, the sticks are characterized by a particularly creamy texture, even after repeated use.

In a further very particularly preferred embodiment, and antiperspirants stick according to the invention is therefore characterized in that it includes at least one alkoxy-modified silicone (A) having a molar mass of from 800 to 10,000 g/mol, preferably from 1,000 to 9,000 g/mol, more preferably from 2,000 to 8,000 g/mol, and particularly preferably from 2,500 to 5,000 g/mol.

Particularly well-suited silicones are for example:

Abil B 8843 from the company Evonic, PEG-14 DIMETHICONE
Xiameter OFX 0193 Fluid from the company Dow Corning, PEG-12 Dimethicone

As second essential ingredient (B), the antiperspirant sticks according to the invention include at least one ester of a C₈-C₁₄ alkanoic acid and a C₈-C₁₄ alkanol.

The C₈-C₁₄ alkanoic acids are linear alkanoic acids. Examples of preferred C₈-C₁₄ alkanoic acids are caprylic acid (1-octanoic acid) C₇H₁₅COOH, pelargonic acid (1-nonanoic acid) C₈C₁₇COOH, capric acid (1-decanoic acid) C₉C₁₉COOH, 1-undecanoic acid C₁₀H₂₁COOH, lauric acid (1-dodecanoic acid) C₁₁H₂₃COOH, 1-tridecanoic acid C₁₂H₂₅COOH and myristic acid (1-tetradecanoic acid) C₁₃H₂₇COOH.

Myristic acid is particularly preferred.

The C₈-C₁₄ alkanols are linear alkanols. C₈-C₁₄ alkanols according to the invention are 1-otanol, 1-nonanol, 1-decanol, 1-undecanol, 1-dodecanol (lauryl alcohol), 1-tridecanol and 1-tetradecanol (myristyl alcohol).

Myristyl alcohol is particularly preferred.

The esters of a C₈-C₁₄ alkanoic acid and a C₈-C₁₄ alkanol are preferably compounds of the following structure (B1):

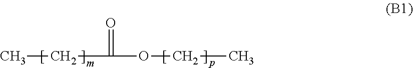

in which
m stands for an integer from 6 to 12, preferably for an integer from 8 to 12, and particularly preferably for an integer from 10 to 12, and
p stands for an integer from 7 to 13, preferably for an integer from 9 to 13, and particularly preferably for an integer from 11 to 13.

In a further very particularly preferred embodiment, and antiperspirants stick according to the invention is therefore characterized in that it includes an ester (B) of formula (B1)

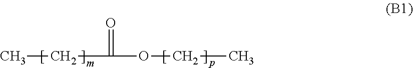

in which
m stands for an integer from 6 to 12, preferably for an integer from 8 to 12, and particularly preferably for an integer from 10 to 12, and
p stands for an integer from 7 to 13, preferably for an integer from 9 to 13, and particularly preferably for an integer from 11 to 13.

It has surprisingly proven to be particularly advantageous if the esters (B) are produced by esterification of an alkanoic acid with an alcohol of the same chain length. In other words, particularly good results were observed in respect of creaminess and release of the antiperspirant active ingredient if the numbers m and p in the esters of formula (B1) corresponded to the formula

m+1=p.

In a further very particularly preferred embodiment, and antiperspirant stick according to the invention is therefore characterized in that it includes an ester (B) of formula (B1) in which the numbers m and p satisfy the following equation:

m+1=p.

Particularly preferred esters (B) are selected from the group 1-octanoic acid (1-octyl ester), 1-nonanoic acid (1-nonyl ester), 1-decanoic acid (1-decyl ester), 1-undecanoic acid (1-undecyl ester), 1-dodecanoic acid (1-dodecyl ester), 1-tridecanoic acid (1-tridecyl ester) and 1-tetradecanoic ester (1-tetradecyl ester (alternative name: myristyl myristate).

1-tetradecanoic acid-1-tetradecyl ester (alternative name: myristyl myristate, tetradecyl tetradecanoate) having the CAS number 3234-85-3 is explicitly very particularly preferred.

In a further embodiment, what is particularly preferred is an antiperspirant stick including
a) at least one non-ionic alkoxy-modified silicone,
b) 1-tetradecanoic acid-1-tetradecyl ester (myristyl myristate), and
c) at least one antiperspirant active ingredient from the group of aluminum salts or aluminum-zirconium salts.

In a further embodiment, what is particularly preferred is an antiperspirant stick including
a) PEG-12 dimethicone,
b) 1-tetradecanoic acid-1-tetradecyl ester (myristyl myristate), and
c) at least one antiperspirant active ingredient from the group of aluminum salts or aluminum-zirconium salts.

The antiperspirant sticks according to the invention are produced with a low water content or preferably in anhydrous form. The term “with a low water content” is understood so that the antiperspirant compositions include 2.5 to at most 10% by weight of free water, in relation to the total weight of the stick. The term “anhydrous” is understood in accordance with the invention so that the compositions include 0 to at most 2.5% by weight, preferably 0 to at most 1.0% by weight of free water, in relation to the total weight of the stick. The content of crystal water, hydration water or similarly molecularly bonded water, which can be included in the used constituents, in particular in optionally included antiperspirant active ingredients, does not constitute free water in the sense of the present application.

In a further very particularly preferred embodiment, and antiperspirants stick according to the invention is therefore characterized in that it has a water content of at most 10.0% by weight, preferably of at most 7.5% by weight, more preferably of at most 5.0% by weight, even more preferably of at most 2.5% by weight, and particularly preferably of at most 1.0% by weight, in relation to the total weight of the stick.

The total weight of the stick, in this context, is understood to mean the total weight of the antiperspirant composition present in the stick shape, conical shape or cylinder shape (without consideration of any material used for packaging or for wrapping).

The objective of the present invention is to minimize or prevent the crusting or hardening that occurs with repeated use of the stick. Even after repeated use, the stick should still have a creamy consistency. This objective is achieved by adding a combination of alkoxy-modified silicone (A) and ester of s C8-C14 alkanoic acid and a C8-C14 alkanol (B) to the wax matrix of the stick.

In other words, by adding the two active ingredients (A) and (B), the hydrophobicity of the wax matrix is reduced to such an extent that the antiperspirant active ingredients (C) can dissolve significantly in the water phase (i.e. the sweat), but at the same time and excessive infiltration of water into the wax matrix is prevented. By adding the active ingredients (A) and (B), the hydrophobicity of the wax matrix is therefore set in a defined manner to the optimal value. An optimal setting of the hydrophobicity is in particular possible if the active ingredients (A) and (B) are used in specific quantity ranges.

The formation of the hardening or crusting caused by the use of the stick could be particularly reduced or even prevented if one or more alkoxy-modified silicones (A) were used in a total amount of from 0.5 to 10% by weight, preferably from 1.0 to 8.0% by weight, more preferably from 1.8 to 7.5% by weight, and particularly preferably from 2.1 to 5.2% by weight, in relation to the total weight of the stick.

In a further very particularly preferred embodiment, an antiperspirant stick according to the invention is therefore characterized in that it includes one or more alkoxy-modified silicones (A) in a total amount of from 0.5 to 10% by weight, preferably from 1.0 to 8.0% by weight, more preferably from 1.8 to 7.5% by weight, and particularly preferably from 2.1 to 5.2% by weight, in relation to the total weight of the stick.

The creamy consistency of the stick was furthermore in particular still present even after repeated use if the stick included one or more esters (B) in a total amount of from 0.5 to 5.0% by weight, preferably from 1.0 to 4.0% by weight, more preferably from 1.3 to 3.5% by weight, and particularly preferably from 1.6 to 2.5% by weight, in relation to the total weight of the stick.

In a further very particularly preferred embodiment, an antiperspirant stick according to the invention is therefore characterized in that it includes one or more esters (B) in a total amount of from 0.5 to 5.0% by weight, preferably from 1.0 to 4.0% by weight, more preferably from 1.3 to 3.5% by weight, and particularly preferably from 1.6 to 2.5% by weight, in relation to the total weight of the stick.

The total weight of the stick in this context is again understood to mean the total weight of the antiperspirant composition present in the stick shape, conical shape or cylinder shape (without consideration of any material used for packaging or for wrapping).

When setting the hydrophobicity of the wax matrix, the active ingredients of the two active ingredient groups (A) and (B) interacts and have a mutual influence on one another. Here, it has in particular proven to be advantageous if the silicone(s) (A) and the ester(s) (B) are used either in equal quantities, or if the silicone(s) (A) are used in an excess in relation to the ester(s) (B). It was particularly advantageous if the ratio by weight of all alkoxy-modified silicones (A) included in the stick to all esters (B) included in the stick, i.e. the ratio by weight of (A):(B) was set to a value of from 1.0 to 4.2, preferably from 1.1 to 4.0, more preferably from 1.6 to 3.2, and particularly preferably from 1.8 to 2.4.

In a further very particularly preferred embodiment, an antiperspirant stick according to the invention is therefore characterized in that the ratio by weight of all alkoxy-modified silicones (A) included in the stick to all esters (B) included in the stick, i.e. the ratio by weight of (A):(B) lies at a value of from 1.0 to 4.2, preferably from 1.1 to 4.0, more preferably from 1.6 to 3.2, and particularly preferably from 1.8 to 2.4.

### Example: An Antiperspirant Stick Includes

a) 4.0% by weight of PEG-12 dimethicone
b) 1.9% by weight of myristyl myristate
c) 17.8% by weight of aluminum zirconium trichlorohydrex gly (AAZG 531 Summit Reheis)
   further ingredients: to 100% by weight
   The ratio by weight of (A):(B) is (4.0/1.9)=2.1.

As third ingredient (C) essential to the invention, the antiperspirant sticks of the present invention include at least one antiperspirant active ingredient from the group of aluminum salts or aluminum-zirconium salts.

The antiperspirant aluminum salts are preferably selected from the water-soluble astringent inorganic and organic salts of aluminum and aluminum-zirconium mixtures. Aluminosilicates and zeolites in accordance with the invention are not classed as antiperspirant active ingredients.

In accordance with the invention, water solubility is understood to mean a solubility of at least 3% by weight at 20° C., in other words that amounts of at least 3 g of the antiperspirant active ingredient are soluble in 97 g of water at 20° C.

Particularly preferred antiperspirant active ingredients are selected from aluminum chlorohydrate, in particular aluminum chlorohydrate with the general formula [Al₂(OH)₅Cl.1-6H₂O]ₙ, preferably [Al₂(OH)₅Cl.2-3H₂O]ₙ, which can be present in the non-activated or in the activated (depolymerised) form, and also aluminum chlorohydrate with the general formula [Al₂(OH)₄Cl₂.1-6H₂O]ₙ, preferably [Al₂(OH)₄Cl₂.2-3H₂O]ₙ, which can be present in the non-activated or in the activated (depolymerised) form.

Also preferred are aluminum sesquichlorohydrate, aluminum dichlorohydrate, aluminum chlorohydrex propylene glycol (PG) or aluminum chlorohydrex polyethylene glycol (PEG), aluminum or aluminum-zirconium glycol complexes, for example aluminum or aluminum-zirconium propylene glycol complexes, aluminum sesquichlorhydrex PG or aluminum sesquichlorohydrex PEG, aluminum PG dichloorhydrex or aluminum PEG dichlorohydrex, aluminum hydroxide, furthermore selected from the aluminum-zirconium chlorohydrates such as aluminum-zirconium trichlorohydrate, aluminum-zirconium tetrachlorohydrate, aluminum-zirconium pentachlorohydrate, aluminum-zirconium octachlorohydrate, the aluminum-zirconium chlorohydrate glycine complexes such as aluminum-zirconium trichlorohydrex glycine, aluminum-zirconium tetrachlorohydrex glycine, aluminum-zirconium pentachlorohydrex glycine, aluminum-zirconium octachlorohydrex glycine, potassium aluminum sulfate (KAI(SO₄)₂.12H₂O, Alaum), aluminum undecylenoyl collagen amino acid, sodium aluminum lactate+aluminum sulfate, sodium aluminum chlorohydroxy lactate, aluminum bromohydrate, aluminum chloride, aluminum salts of lipoamino acids, aluminum sulfate, aluminum lactate, aluminum chloro hydroxy allantoin and sodium aluminum chlorohydroxy lactate.

Antiperspirant active ingredients that are particularly preferred in accordance with the invention are selected from what are known as “activated” aluminum salts and aluminum-zirconium salts, which are also referred to as antiperspirant active ingredients having enhanced activity. Active ingredients of this type are known in the prior art and are also commercially available. Activated aluminum salts and aluminum-zirconium salts are generally produced by heat treatment of a relatively diluted solution of the salt (for example approximately 10% by weight salt) in order to increase the HPLC peak 4-to-peak 3 area ratio thereof. The activated salt may then be dried to a powder, especially spray-dried. In addition to the spray drying, roller drying is also suitable for example.

Activated aluminum salts and aluminum-zirconium salts typically have an HPLC peak 4-to-peak 3 area ratio of at least 0.4, preferably of at least 0.7, particularly preferably at least 0.9, wherein at least 70% of the aluminum is to be attributed to these HPLC peaks.

Activated aluminum salts and aluminum-zirconium salts do not necessarily have to be used in the form of a spray-dried powder. Antiperspirant active ingredients that are also preferred in accordance with the invention are non-aqueous solutions or solubilizates of an activated antiperspirant aluminum salts or aluminum-zirconium salts, which are stabilized against the loss of activation, i.e. against the quick reduction of the HPLC peak 4:peak 3 area ratio of the salt, by the addition of an effective amount of a polyvalent alcohol having 3 to 6 carbon atoms and 3 to 6 hydroxyl groups, preferably propylene glycol, sorbitol and pentaerythritol.

Compositions which include in % by weight (USP): 18-45% by weight of an activated aluminum salt or aluminum-zirconium salt, 55-82% by weight of at least one anhydrous polyvalent alcohol having 3 to 6 carbon atoms and 3 to 6 hydroxyl groups, preferably 1,2-propylene glycol, 1,3-butylene glycol, diethylene glycol, dipropylene glycol, glycerol, sorbitol and pentaerythritol, particularly preferably 1,2-propylene glycol, are preferred, for example.

Complexes of activated antiperspirant aluminum salts or aluminum-zirconium salts with a polyvalent alcohol including 20-50% by weight, preferably 20-42% by weight, of activated antiperspirant aluminum salt or aluminum-zirconium salt and 2-16% by weight of molecularly bound water, wherein the balance to 100% by weight is at least one polyvalent alcohol having 3 to 6 carbon atoms and 3 to 6 hydroxyl groups, are also particularly preferred. Propylene glycol, propylene glycol/sorbitol mixtures and propylene glycol/pentaerythritol mixtures are preferred such alcohols.

Further preferred antiperspirant active ingredients are basic calcium-aluminum salts. These salts are produced by reacting calcium carbonate with aluminum chlorohydroxide or aluminum chloride and aluminum powder or by adding calcium chloride dihydrate to aluminum chlorohydroxide

Further preferred antiperspirant active ingredients are aluminum-zirconium complexes which are buffered with salts of amino acids, in particular with alkaline and alkaline earth glycinates.

Further preferred antiperspirant active ingredients are activated aluminum salts or aluminum-zirconium salts including 5-78% by weight (USP) of an activated antiperspirant aluminum salt or aluminum-zirconium salt, and amino acid, or hydroxyalkanoic acid in such an amount as to provide a (amino acid or hydroxyalkanoic acid) to (Al+Zr) ratio by weight of 2:1-1:20, preferably 1:1 to 1:10, and also a water-soluble calcium salt in such an amount as to provide a Ca:(Al+Zr) ratio by weight of 1:1-1:28 and preferably 1:2-1:25. Particularly preferred solid activated antiperspirant salt compositions include 48-78% by weight (USP), preferably 66-75% by weight of an activated aluminum salt or aluminum-zirconium salt, and 1-16% by weight, preferably 4-13% by weight of molecularly bound water (hydration water), and also such an amount of water-soluble calcium salt that the Ca:(Al+Zr) ratio by weight is 1:1-1:28, preferably 1:2-1:25, and such an amount of amino acid that the amino acid to (Al+Zr) ratio by weight is 2:1-1:20, preferably 1:1 to 1:10.

Further particularly preferred solid antiperspirant activated salt compositions include 48-78% by weight (USP), preferably 66-75% by weight of an activated aluminum salt or aluminum-zirconium salt, and 1-16% by weight, preferably 4-13% by weight of molecularly bound water (hydration water), and also such an amount of water-soluble calcium salt that the Ca:(Al+Zr) ratio by weight is 1:1-1:28, preferably 1:2-1:25, and such an amount of glycine that the glycine to (Al+Zr) ratio by weight is 2:1-1:20, preferably 1:1 to 1:10.

Further particularly preferred solid antiperspirant activated salt compositions include 48-78% by weight (USP), preferably 66-75% by weight of an activated aluminum salt or aluminum-zirconium salt, and 1-16% by weight, preferably 4-13% by weight of molecularly bound water, and also such an amount of water-soluble calcium salt that the Ca:(Al+Zr) ratio by weight is 1:1-1:28, preferably 1:2-1:25, and such an amount of hydroxyalkanoic acid that the hydroxyalkanoic acid to (Al+Zr) ratio by weight is 2:1-1:20, preferably 1:1 to 1:10.

For the stabilization of the antiperspirant salts, preferred amino acids are selected from glycine, alanine, leucine, isoleucine, β-alanine, valine, cysteine, serine, tryptophan, phenylalanine, methionine, β-amino-n-butanoic acid and γ-amino-n-butanoic acid and salts thereof, in each case in the d-form, the 1-form an the dl-form; glycine is particularly preferred.

For the stabilization of the antiperspirant salts, preferred hydroxyalkanoic acids are selected from glycolic acid and lactic acid.

Further preferred activated aluminum salts are those of general formula Al₂(OH)₆₋ₐXa, in which X is Cl, Br, I or NO₃ and “a” is a value from 0.3 to 5, preferably from 0.8 to 2.5, and particularly preferably is 1 to 2, such that the molar ratio of Al:X is 0.9:1 to 2.1:1. A little hydration water is generally associatively bound in these salts, typically 1 to 6 mol water per mol salt. Aluminum chlorohydrate is particularly preferred (i.e. X is Cl in the aforementioned formula) and especially ⅚ basic aluminum chlorohydrate, in which “a” is 1, such that the molar ratio of aluminum to chlorine is 1.9:1 to 2.1:1.

Preferred activated aluminum-zirconium salts are those which constitute mixtures or complexes of the above-described aluminum salts with zirconium salts of formula ZrO(OH)_{2-pb}Y_{b}, in which Y is Cl, Br, I, NO₃ or SO₄, b is a rational number from 0.8 to 2, and p is the valency of Y, as are disclosed for example in U.S. Pat. No. 6,074,632. The zirconium salts generally also have a little hydration water associative Lee bound, typically 1 to 7 mol water per mol salt. The zirconium salt is preferably zirconyl hydroxychloride with the formula ZrO(OH)_{2-b}Cl_{b}, in which b is a rational number from 0.8 to 2, preferably is 1.0 to 1.9. Preferred aluminum-zirconium salts have an Al:Zr molar ratio from 2 to 10 and a metal:(X+Y) ratio of from 0.73 to 2.1, preferably 0.9 to 1.5. A particularly preferred salt is an aluminum-zirconium chlorohydrate (i.e. X and Y are Cl) which has an Al:Zr ratio from 2 to 10 and a molar metal:Cl ratio from 0.9 to 2.1. The term aluminum-zirconium chlorohydrate comprises the tri-, tetra-, penta- and octachlorohydrate forms.

Preferred antiperspirant aluminum-zirconium salts have a molar metal-to-chloride ratio of 0.9-1.5, preferably 0.9-1.3, particularly preferably 0.9-1.1.

Zirconium-free aluminum salts that are particularly preferred in accordance with the invention have a molar metal-to-chloride ratio of 1.9-2.1. Zirconium-free aluminum sesquichlorohydrates that are particularly preferred in accordance with the invention have a molar metal-to-chloride ratio of 1.5:1-1.8:1. Preferred aluminum-zirconium chlorohydrates generally have the empirical formula AlₙZr(OH)_{[3n+4-m(n+1)]}(Cl)_{[m(n+1)]} with n=2.0-10.0, preferably 3.0-8.0, m=0.77-1.11 (corresponding to a molar metal (Al+Zr)-to-chloride ratio of 1.3-0.9), preferably m=0.91-1.11 (corresponding to M:Cl=1.1-0.9), and particularly preferably m=1.00-1.11 (corresponding to M:Cl=1.0-0.9), furthermore very preferably m=1.02-1.11 (corresponding to M:Cl=0.98-0.9) and also very preferably m=1.04-1.11 (corresponding to M:Cl=0.96-0.9). A little hydration water is generally associatively bound in these salts, typically 1-6 mol water per mol salt, corresponding to 1-16% by weight, preferably 4-13% by weight of hydration water.

The preferred aluminum-zirconium chlorohydrates are usually associated with an amino acid in order to prevent the polymerization of the zirconium species during the production process. Preferred stabilizing amino acids are selected from glycine, alanine, leucine, isoleucine, β-alanine, cysteine, valine, serine, tryptophan, phenylalanine, methionine, β-amino-n-butanoic acid and γ-amino-n-butanoic acid and salts thereof, in each case in the d-form, the l-form and the dl-form; glycine is particularly preferred. The amino acid is included in an amount of 1-3 mol, preferably 1.3-1.8 mol, in each case per mol zirconium in the salt.

Preferred antiperspirant salts are aluminum-zirconium tetrachlorohydrates (Al:Zr=2-6; M:Cl=0.9-1.3), in particular salts having a molar metal-to-chloride ratio of 0.9-1.1, preferably 0.9-1.0.

What are also preferred in accordance with the invention are aluminum-zirconium chlorohydrate glycine salts which are stabilized with betaine ((CH₃)₃N⁺—CH₂—COO⁻). Particularly preferred corresponding compounds have a molar total (betaine+glycine)/Zr ratio of (0.1-3.0):1, preferably (0.7-1.5):1, and a molar ratio of betaine to glycine of at least 0.001:1.

In a particularly preferred embodiment according to the invention, what is known as an “activated” salt is included is particularly effective antiperspirant salt, in particular one with a high HPLC peak 5 aluminum content, in particular with a peak 5 area of at least 33%, particularly preferably at least 45%, in relation to the total area under peaks 2-5, measured by HPLC of a 10% by weight aqueous solution of the active ingredient under conditions in which the aluminum species are dissolved in at least four successive following peaks (denoted peaks 2-5). Preferred aluminum-zirconium salts are those having a high HPLC peak 5 aluminum content (also referred to as “E⁵AZCH”).

Activated “E⁵AZCH” salts of which the HPLC peak 4-to-peak 3 area ratio peak is at least 0.4, preferably at least 0.7, particularly preferably at least 0.9 are also preferred. Further particularly preferred antiperspirant active ingredients are aluminum-zirconium salts having a high HPLC peak 5 aluminum content which additionally are stabilized with a water-soluble strontium salt and/or with a water-soluble calcium salt.

The formulation of the agents according to the invention in a specific administration form, for example in antiperspirant roll-on or an antiperspirant stick, or antiperspirant gel is preferably directed towards the requirements of the intended purpose.

The antiperspirant stick can include the antiperspirant active ingredient(s), in particular the aforementioned preferred and particularly preferred representatives, (C) in a total amount of from 5.0 to 20.0% by weight, preferably from 7.0 to 17.5% by weight, more preferably from 8.5 to 15.5% by weight, and particularly preferably from 11.5 to 14.5% by weight, in relation to the total weight of the stick.

In a further very particularly preferred embodiment, an auntie person and stick according to the invention is therefore characterized in that it includes one or more antiperspirant active ingredients (C) in a total amount of from 5.0 to 20.0% by weight, preferably from 7.0 to 17.5% by weight, more preferably from 8.5 to 15.5% by weight, and particularly preferably from 11.5 to 14.5% by weight, in relation to the total weight of the stick.

For an optimized active ingredient release, the antiperspirant active ingredient(s) (C) to the sum of alkoxy-modified silicones (A) and esters (B) are also used in a specific ratio to one another. A particularly good reduction of sweat could be observed if the ratio by weight of all antiperspirant active ingredients (C) included in the stick to the sum of the alkoxy-modified silicones (A) and esters (B) included in the stick, i.e. the ratio by weight of (C)/[(A)+(B)], was at a value of from 1.0 to 4.5, preferably from 1.5 to 3.5, more preferably from 1.8 to 3.1, and particularly preferably from 1.9 to 2.5.

In a further very particularly preferred embodiment, an antiperspirant stick according to the invention is therefore characterized in that the ratio by weight of all antiperspirant active ingredients (C) included in the stick to the sum of the alkoxy-modified silicones (A) and esters (B) included in the stick, i.e. the ratio by weight of (C)/[(A)+(B)], is at a value of from 1.0 to 4.5, preferably from 1.5 to 3.5, more preferably from 1.8 to 3.1, and particularly preferably from 1.9 to 2.5.

Example: An antiperspirant stick includes
a) 4.0% by weight of PEG-12 dimethicone
b) 1.9% by weight of myristyl myristate
c) 17.8% by weight of aluminum zirconium trichlorohydrex gly (Summit Reheis)
   (content of active substance=78.5%, content of active substance=13.97% by weight) further ingredients: to 100% by weight
   The ratio by weight of (C)/[(A)+(B)]=[13.97/(1.9+4.0)]=2.37.

Surprisingly, the use of one or more polyethylene glycols could improve the consistency of the antiperspirant sticks further still.

Polyethylene glycol is understood to mean a compound of general formula (D)

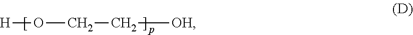

wherein p represents an integer from 2 to 500, preferably from 2 to 250, more preferably from 2 to 200, and particularly preferably from 2 to 150. The polyethylene glycol(s) is/are included in the antiperspirant stick preferably in a total amount of from 0.01 to 10.0% by weight, preferably from 0.07 to 7.0% by weight, more preferably from 0.15 to 4.5% by weight, and particularly preferably from 0.25 to 1.0% by weight, in relation to the total weight of the stick.

In a further very particularly preferred embodiment, and antiperspirant stick according to the invention is therefore characterized in that it additionally includes one or more polyethylene glycols in a total amount of from 0.01 to 10.0% by weight, preferably from 0.07 to 7.0% by weight, more preferably from 0.15 to 4.5% by weight, and particularly preferably from 0.25 to 1.0% by weight, in relation to the total weight of the stick.

The antiperspirant sticks according to the invention include the active ingredients essential to the invention (A), (B) and (C) preferably in a lipid or wax matrix. The lipid or wax matrix of the stick compositions according to the invention can comprise at least one lipid or wax component having a melting point >50° C. Generally, waxes of firm to set hard consistency, have a rough to finally crystal lean structure, are color-translucent to opaque, but not glass-like, and melt above 50° C. without decomposition. They have a low viscosity already just above the melting point and exhibit a strongly temperature-dependent consistency and solubility.

By way of example, natural plant waxes are preferred in accordance with the invention, for example candelilla wax, carnauba wax, Japan wax, sugar cane wax, ouricouri wax, cork wax, sunflower wax, fruit waxes such as orange wax, lemon wax, grapefruit wax, and animal waxes, for example beeswax, shellac wax, and spermaceti. In the sense of the invention, it can be particularly preferred to use hydrogenated or hardened waxes. Chemically modified waxes, in particular hard waxes, such as montan ester waxes, hydrogenated jojoba waxes and sasol waxes, can also be used as wax component. These synthetic waxes, which are also preferred in accordance with the invention, for example include polyalkylene wax and also alkyl and alkyl aryl esters of dimer fatty acids.

A wax component which can be included in the antiperspirant stick in addition to the esters (B) according to the invention is selected from at least one ester of a saturated, monovalent C₁₆-C₆₀ alcohol and a saturated C₁₆-C₃₆ monocarboxylic acid.

In accordance with the invention, these also include lactides, i.e. the cyclic double esters of α-hydroxycarboxylic acids of the corresponding chain length. The esters are composed of saturated, branched or unbranched monocarboxylic acids and saturated, branched or unbranched monovalent alcohols. Esters of aromatic carboxylic acids or hydroxycarboxylic acids (for example (12-hydroxystearic acid) and saturated, branched or unbranched alcohols can be used in accordance with the invention, provided the wax component has a melting point >50° C. These wax components can be selected from the group of esters of saturated, branched or unbranched alkanoic carboxylic acids with a chain length of 16 to 24 C atoms and the saturated, branched or unbranched alcohols with a chain length of 16 to 50 C atoms, which have a melting point >50° C.

In particular, C₁₆₋₃₆ alkyl stearates and C₁₈₋₃₈ alkyl hydroxy stearoyl stearates, C₂₀₋₄₀ alkyl erucates, and cetearyl behenate can be advantageous as wax component. The wax or the wax components have a melting point >50° C., preferably >60° C.

A particularly preferred embodiment of the invention includes a C₂₀-C₄₀ alkyl stearate as wax component. This ester is known under the name Kesterwachs® K82H or Kesterwachs® K80H and is sold by Koster Keunen Inc. it is the synthetic imitation of the monoester fraction of beeswax and is characterized by its hardness, its oil gelling capability broad compatibility with lipid components. This wax can be used as stabilizer and consistency regulator for W/O and O/W emulsions. Kesterwachs offers the advantage that it has an excellent oil gelling capability, even at low concentrations, and therefore does not make the stick mass too heavy and enables the mass to be rubbed off in a velvety manner. A further particularly preferred embodiment of the invention includes cetearyl behenate as wax component, i.e. mixtures of cetyl behenate and stearyl behenate. This ester is known under the name Kesterwachs® K62 and is sold by Koster Keunen Inc.

Further preferred lipid or wax components having a melting point >50° C. are the triglycerides of saturated and optionally hydroxylated C₁₂₋₃₀ fatty acids, such as hardened triglyceride fats (hydrogenated palm oil, hydrogenated coconut oil, hydrogenated castor oil), glyceryl tribehenate (Tribehenin) or glyceryl tri-12-hydroxystearate, also synthetic full esters of fatty acids and glycols or polyols having 2-6 carbon atoms, provided they have a melting point above 50° C., for example preferably C₁₈-C₃₆ acid triglycerides (Syncrowax® HGL-C).

Hydrogenated castor oil obtainable for example as a trade product Cutina® HR, is particularly preferred in accordance with the invention as wax component. Further preferred lipid or wax components having a melting point >50° C. are the saturated linear C₁₄-C₃₆ carboxylic acids, in particular myristic acid, palmitic acid, stearic acid, and behenic acid and also mixtures of these compounds, for example Syncrowax® AW 1C (C₁₈-C₃₆ fatty acids) or Cutina® FS 45 (palmitic and stearic acid).

More or less all saturated linear fatty acids and fatty alcohols having 14-22 C atoms, triglycerides of saturated fatty acids having 14-22 C atoms, saturated linear wax esters (fatty acid fatty alcohol esters) having 28-44 C atoms, or mixtures therefore are suitable as consolidating fat or wax component. A mixture of 1 part by weight of hydrogenated castor oil and 2-4 parts by weight of stearyl alcohol is very particularly well suited.

The antiperspirant sticks can additionally also include further emulsifiers, such as oil-in-water emulsifiers.

The non-ionic oil-in-water emulsifier is particularly preferably selected from surfactant substances having an HLB value of more than 7, selected from:
ethoxylated C₈-C₂₄ alkanols having on average 10-100 mol ethylene oxide per mol,
ethoxylated C₈-C₂₄ carboxylic acids having on average 10-100 mol ethylene oxide per mol,
alkylmono- and -oligoglycosides having 8 to 22 carbon atoms in the alkyl group and ethoxylated analogues thereof,
ethoxylated sterols,
partial esters of polyglycerols with n=2 to 10 glycerol units and with 1 to 4 saturated or unsaturated, linear or branched, optionally hydroxylated C₈-C₃₀ fatty acid groups, esterified if they have an HLB value of more than 7,
and mixtures of the aforementioned substances.

Preferred antiperspirant sticks according to the invention are characterized in that the non-ionic oil-in-water emulsions are selected from ethoxylated C8-C24 alkanols having on average 10-100 mol ethylene oxide per mol, ethoxylated C8-C24 carboxylic acids having on average 10-100 mol ethylene oxide per mol, alkylmono- and -oligoglycosides having 8 to 22 carbon atoms in the alkyl group and ethoxylated analogues thereof, ethoxylated sterols, partial esters of polyglycerols with n=2 to 10 glycerol units and with 1 to 4 saturated or unsaturated, linear or branched, optionally hydroxylated C₈-C₃₀ fatty acid esters, esterified if they have an HLB value of more than 7, and mixtures of the aforementioned substances. The exthoxylated C₈-C₂₄ alkanols have the formula R¹O(CH₂CH₂O)ₙH, wherein R¹ stands for a linear or branched alkyl group and/or alkenyl group having 8-24 carbon atoms and n, the mean number of ethylene oxide units per molecule, stands for numbers of 10-100, preferably 10-30 mol ethylene oxide with 1 mol capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and also technical mixtures thereof. Adducts of 10-100 mol ethylene oxide with technical fatty alcohols having 12-18 carbon atoms, such as coconut, palm, palm kernel or tallow fatty alcohol, are suitable.

The ethoxylated C₈-C₂₄ carboxylic acids have the formula R¹O(CH₂CH₂O)ₙH, wherein R¹O stands for a linear or branched saturated or unsaturated acyl group having 8-24 carbon atoms, and n, the mean number of ethylene oxide units per molecule, stands for numbers of 10-100, preferably 10-30 mol ethylene oxide with 1 mol caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselinic acid, arachyic acid, gadoleic acid, behenic acid, erucic acid and brassidic acid and also technical mixtures thereof. Adducts of 10-100 mol ethylene oxide with technical fatty acids having 12-18 carbon atoms, such as coconut, palm, palm kernel or tallow fatty acid, are suitable. Particularly preferred are PEG-50 monostearate, PEG-100 monostearate, PEG-50 monooleate, PEG-1 monooleate, PEG-50 monolaurate and PEG-100 monolaurate. The C₁₂-C₁₈ alkanols or the C₁₂-C₁₈ carboxylic acids each having 10-30 units ethylene oxide per molecule, and also mixtures of these substances, in particular Ceteth-12, Ceteth-20, Ceteth-30, Stearaeth-12, Steareth-20, Steareth-30, Laureth-12 and Beheneth-20 are particularly preferably used. C₈-C₂₂ alkylmono- and -oligoglycosides are also preferably used. C₈-C₂₂ alkylmono- and -oligoglycosides represent known commercially available surfactants and emulsifiers. They are produced in particular by reacting glucose or oligosaccharides with primary alcohols having 8-22 carbon atoms. With regard to the glycoside group, it is true that both monoglycosides in which a cyclic sugar group is gycosidically bound to the fatty alcohol and oligomeric glycosides having a degree of oligomerization up to approximately 8, preferably 1-2, are suitable. Here, the degree of oligomerization is a statistical mean value forming the basis of a homologue distribution conventional for such technical products. Products which are obtainable under the trade name Plantacare® include a glucosidically bound C₈-C₁₆ alkyl group with an oligoglucoside group of which the mean degree of oligomerization is 1-2. Particularly preferred C₈-C₂₂ alkylmono- and -oligoglycosides are selected from octylglucoside, decylglucoside, laurylglucoside, palmitylglucoside, isostearylglucoside, stearylglucoside, arachidylglucoside and behenylglucoside and also mixtures thereof. The acylglucamides derived from glucamine are suitable as non-ionic oil-in-water emulsifiers.

Ethoxylated sterols, in particular ethoxylated soya sterols, also represent oil-in-water emulsifiers that are suitable in accordance with the invention. The degree of ethoxylation should be greater than 5, preferably at least 10, so as to have an HLB value greater than 7. Suitable trade products are, for example, PEG-10 Soy Sterol, PEG-16 Soy Sterol, and PEG-25 Soy Sterol.

Partial esters of polyglycerols having 2 to 10 glycerol units and having 1 to 4 saturated or unsaturated, linear or branched, optionally hydroxylated C₈-C₃₀ fatty acid esters are preferably also used, esterified, if they have an HLB value of ore than 7. Diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristat, decaglycerol monoisostearate, decaglycerol monostearate, decaglycerol monooleate, decaglycerol monohydroxystearate, decaglycerol dicaprylate, decaglycerol dicaprate, deca-glycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate and decaglycerol trihydroxystearate are particularly preferred.

The antiperspirant sticks can additionally also include further volatile silicone oils and volatile non-silicone oils.

Particularly preferred volatile oils are volatile silicone oils and volatile non-silicone oils. Volatile silicone oils, which can be cyclic, such as octamethylcyclotetrasiloxane, decamethylcyclopentasiloxane and mixtures thereof, as are included for example in trade products DC 244, 245, 344 and 345 from Dow Corning, are preferred in accordance with the invention. Volatile linear silicone oils are also particularly preferred, in particular hexamethyldisiloxane (L₂), octamethyltrisiloxane (L₃), decamethyltetrasiloxane (L₄) and any mixtures of two and three of L₂, L₃ and/or L₄, preferably mixtures as are included for example in the trade products DC 2-1184, Dow Corning® 200 (0.65 cSt) and Dow Corning® 200 (1.5 cSt) from Dow Corning.

Volatile silicone oils are outstandingly suitable in accordance with the invention since they provide the composition according to the invention with a pleasant feel against the skin and a low potential for soiling clothing. Compositions that are particularly preferred in accordance with the invention are therefore characterized by a content of at least one volatile silicone oil. Here, it is again preferred if the volatile silicone oils, in relation to the weight of the composition, are included in the composition according to the invention in a total amount of from 20 to 80% by weight, in particular from 30 to 80% by weight, particularly preferably from 40 to 75% by weight, very particularly preferably from 45 to 70% by weight.

Besides or instead of the at least one volatile silicone oil, at least one volatile non-silicone oil can also be included. Preferred volatile non-silicone oils are selected from C₈-C₁₆ isoparaffins, in particular from isodecane, isododecane, isotetradecane and isohexadecane and mixtures thereof. Preferably C10-13 isoparaffin (for example trade product Pioner 2094 from Hansen & Rosenthal) are particularly suitable.

This at least one volatile non-silicone oil is also preferably included in a total amount of from 20 to 80% by weight, particularly preferably from 30 to 80% by weight, very particularly preferably from 40 to 75% by weight, in each case in relation to the total weight of the composition.

Due to the feel against the skin and the stability of the resultant compositions, silicone oils in the form of volatile oil are particularly preferred compared to isoparaffins.

Besides the aforementioned substances usually referred to as volatile silicone oils and also besides the aforementioned volatile non-silicone oils, the compositions according to the invention can additionally include at least one non-volatile oil, selected from non-volatile silicone oils and non-volatile non-silicone oils.

Preferred non-volatile silicone oils are selected from linear dimethylpolysiloxanes of high molecular weight, commercially obtainable for example under the name Dow Corning® 190, Dow Corning® 200 fluid with kinematic viscosities (25° C.) in the range of 5-100 cSt, preferably 5-50 cSt or also 5-10 cSt, and Baysilon® 350 M (with a kinematic viscosity (25° C.) of approximately 350 cSt). Non-volatile silicone oils that are likewise preferred in accordance with the invention are selected from silicones of formula (Sil-1), wherein x is selected from integers of 1-20, preferably 1-3.

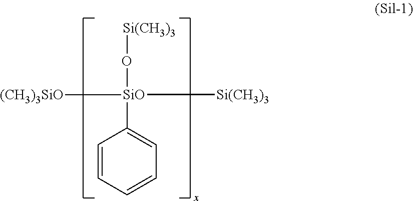

A preferred silicone oil of formula (Sil-1) is obtainable under the INCI name Phenyl Trimethicone.

Natural and synthetic hydrocarbons, such as paraffin oils, C₁₈-C₃₀ isoparaffins, in particular isoeicosan, polyisobutene or polydecene, which for example are obtainable under the name Emery® 3004, 3006, 3010 or under the name Ethylflo® from Albemarle or Nexbase® 2004G from Neste, and also 1,3-di-(2-ethylhexyl) cyclohexane (obtainable for example under the trade names Cetiol®S from Cognis) also belong to the non-volatile non-silicone oils that are preferred in accordance with the invention.

Further non-volatile non-silicone oils that are preferred in accordance with the invention are selected from the benzoic acid esters of linear or branched C₈₋₂₂ alkanols, What are particularly preferred are benzoic acid C12-C15 alkyl esters, for example obtainable as the trade product Finsolv® TN, benzoic acid isostearyl esters, for example obtainable as the trade product Finsolv® SB, ethylhexyl benzoate, for example obtainable as the trade product Finsolv® EB, and benzoic acid octyl dodecyl esters, for example obtainable as the trade product Finsolv® BOD.

Further non-volatile silicone oils that are preferred in accordance with the invention are selected from the triglycerides of linear or branched, saturated or unsaturated, optionally hydroxylated C₈₋₃₀ fatty acids. The use of natural oils, for example soybean oil, cottonseed oil, sunflower oil, palm oil, palm kernel oil, linseed oil, almond oil, castor oil, corn oil, rapeseed oil, olive oil, sesame oil, safflower oil, wheat germ oil, peach kernel oil and the liquid fractions of coconut oil and the like can be particularly suitable. However, synthetic triglyceride oils, in particular Capric/Caprylic Triglycerides, for example the trade products Myritol® 318, Myritol® 331 (Cognis) or Miglyol® 812) (Hüls) with unbranched fatty acid esters and glyceryl triisostearin and the trade products Estol® GTEH 3609 (Uniqema) or Myritol® GTEH (Cognis) with branched fatty acid esters are also suitable.

Further non-volatile non-silicone oils that are particularly preferred in accordance with the invention are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Further non-volatile non-silicone oils that are particularly preferred in accordance with the invention are selected from the addition products of 1 to 5 propylene oxide units with monovalent or polyvalent C₈₋₂₂ alkanols such as octanol, decanol, decanediol, lauryl alcohol, myristyl alcohol and stearyl alcohol, for example PPG-2 myristyl ether and PPG-3 myristyl ether (Witconol® APM).

Further non-volatile non-silicone oils that are particularly preferred in accordance with the invention are selected from the addition products of at least 6 ethylene oxide and/or propylene oxide units with monovalent or polyvalent C₃₋₂₂ alkanols such as glycerol, butanol, butanediol, myristyl alcohol and stearyl alcohol, which can be esterified as desired, for example PPG-14 butyl ether (Ucon Fluid® AP), PPG-9 butyl ether (Breox® B25), PPG-10 butanediol (Macol® 57), PPG-15 stearyl ether (Arlamol® E) and Glycereth-7-diisononanoate.

Further non-volatile non-silicone oils that are particularly preferred in accordance with the invention are selected from the C₈-C₂₂ alkanol esters of monovalent or polyvalent C₂-C₇ hydroxycarboxylic acids, in particular the esters of glycolic acid, lactic acid, malic acid, tartaric acid, citric acid and salicylic acid. Such esters based on linear C_{14/15} alkanols, for example C₁₂-C₁₅ alkyl lactate, and based on C_{12/13} alkanols branched in the 2-position can be purchased under the trade names Cosmacol® from the company Nordmann, Rassmann GmbH & Co, Hamburg, in particular the trade products Cosmacol® ESI, Cosmacol® EMI and Cosmacol® ETI.

Further non-volatile non-silicone oils that are particularly preferred in accordance with the invention are selected from the symmetric, asymmetric or cyclic esters of carbonic acid with alkanols, for example glycerol carbonate, dicaprylyl carbonate (Cetiol® CC) or the esters according to the teaching of DE 19756454.

Further oils which can be preferred in accordance with the invention are selected from the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols.

It is preferred in accordance with the invention is the compositions according to the invention, in relation to the total weight of the composition, include the non-volatile oils in a total amount of from 0 to 20% by weight, in particular from 0 to 10% by weight.

The composition according to the invention particularly preferably includes additionally at least one fatty alcohol. Fatty alcohols are understood in accordance with the invention to mean primary alcohols in the form of monohydroxy compounds of a linear, aliphatic hydrocarbon, wherein said hydrocarbon has at least 12 carbon atoms and apart from the hydroxy group does not carry any further substituents.

It is preferred in accordance with the invention is the fatty alcohols used to prepare the composition according to the invention are present as a solid and all definitions relating to fatty alcohols relate to solid fatty alcohols in the sense of the application and include liquid fatty alcohols exclusive of the oil component of the composition according to the invention.

Preferred compositions according to the invention include 1 to 30% by weight, preferably 1.25 to 27.5% by weight, more preferably 1.5 to 25% by weight, particularly preferably 1.75 to 22.5% by weight and in particular 2 to 20% by weight if C12-C18 alkanols.

It is more preferred to use a mixture of at least one fatty alcohol having 12 to 18 carbon atoms and at least one fatty alcohol having more than 18 carbon atoms, wherein the ratio by weight of the fatty alcohols having 12 to 18 carbon atoms to the rest of the fatty alcohols lies in a ratio by weight range of from 1 to 1 to 2 to 1.

Compositions that are preferred in accordance with the invention have a total amount of fatty alcohol, in each case in relation to the weight of the total composition, of less than 12% by weight, in particular less than 10% by weight. In a composition that is particularly preferred in accordance with the invention, the total amount of fatty alcohols is 3 to 12% by weight, in particular 4 to 10% by weight, in relation to the total weight of the composition.

Further preferred compositions according to the invention are characterized in that they additionally include at least one solid, water-insoluble particulate filler, for example in order to improve the consistency and the sensory properties. In an extremely preferred embodiment, this filler is selected from optionally modified starches (for example from maize, rice, potatoes) and starch derivatives, which are pre-agglutinated as desired (for example Dry FLO PC from the company AKZO). silicon dioxide, silicas, for example Aerosil® types, spherical polyalkylsesquisiloxane particles (in particular Aerosil® 8972 and Aerosil® 200V from Degussa), silica gels, talc, kaolin, magnesium aluminum silicates, boron nitride, lactoglobulin derivatives, for example sodium C₈₋₁₆ isoalkyl succinyl lactoglobulin sulfonate, obtainable from Brooks Industries as trade product Biopol® OE, glass powders, polymer powders, especially of polyolefins, polycarbonates, polyurethanes, polyamides, for example nylon, polyesters, polystyrenes, polyacrylates, (meth)acrylate- or (meth)acrylate-vinylidene copolymers, which may be crosslinked, or silicones, and mixtures of these substances.

Polymer powders based on a polymethacrylate copolymer are obtainable for example as trade product Polytrap® 6603 (Dow Corning). Other polymer powders, for example based on polyamides, are obtainable under the name Orgasol® 1002 (polyamide 6) and Orgasol® 2002 (polyamide 12) from Elf Atochem. Further polymer powders which are suitable as fillers preferred in accordance with the invention are, for example, polymethacrylates (Micropearl® M from SEPPIC or Plastic Powder A from NIKKOL), styrene-divinylbenzene copolymers (Plastic Powder FP from NIKKOL), polyethylene and polypropylene powders (ACCUREL® EP 400 from AKZO) or also silicone polymers (Silicone Powder X2-1605 from Dow Corning).

Preferred compositions according to the invention are characterized in that they include at least one solid, water-insoluble particulate filler in a total amount of from 1-99% by weight, preferably 2-90% by weight, particularly preferably 3-15% by weight, extremely preferably 4, 5, 6, 7, 8, 9, 10, 11, 12, 13 and 14% by weight, in each case in relation to the total composition.

Further preferred compositions according to the invention are therefore characterized in that they additionally include at least one lipophilic thickener.

Lipophilic thickeners that are preferred in accordance with the invention are selected from hydrophobized clay minerals, fumed silicas, ethylene/propylene/styrene copolymers, butylene/ethylene/styrene copolymers, dextrin esters, silicone elastomers, waxes that are solid under normal conditions, and/or glycerol esters. The composition according to the invention particularly preferably additionally includes, as lipophilic thickener, at least one silicone elastomer.

Preferred hydrophobized clay minerals are selected from hydrophobized montmorillonites, hydrophobized hectorites and hydrophobized bentonites, particularly preferably from disteardimonium hectorites, stearalkonium hectorites, Quaternium-18 hectorites and Quaternium-18 bentonites. The commercially available thickeners provide these hydrophobized clay minerals in the form of a gel in an oil component, preferably in cyclomethicone and/or a non-silicone oil component, such as propylene carbonate. For example, such gels are obtainable under the trade name Bentone® or Thixogel.

Compositions that are preferred in accordance with the invention are characterized in that they include at least one hydrophobized clay mineral in a total amount of 0.5-10% by weight, preferably 1-7% by weight, particularly preferably 2-6% by weight, extremely preferably 3-5% by weight, in each case in relation to the total weight of the composition according to the invention.

Further lipophilic thickeners that are preferred in accordance with the invention are selected from fumed silicas, for example the trade products within the Aerosil® range from Evonik Degussa. What are particularly preferred are hydrophobized fumed silicas, particularly preferably Silica Silylate and Silica Dimethyl Silylate.

Compositions that are preferred in accordance with the invention are characterized in that they include at least one fumed silica, preferably at least one (preferably hydrophobized) fumed silica, in a total amount of 0.5-10% by weight, preferably 1-7% by weight, particularly preferably 2-6% by weight, extremely preferably 3-5% by weight, in each case in relation to the total weight of the composition according to the invention.

Further compositions that are preferred in accordance with the invention are characterized in that they include at least one hydrophobized fumed silica and at least one hydrophilic silica.

Further lipophilic thickeners that are preferred in accordance with the invention are selected from ethylene/propylene/styrene copolymers and butylene/ethylene/styrene copolymers. The copolymers are particularly preferably used as pre-thickened oil-based gel. Gels of this type are obtainable for example under the trade name Versagel® (ex Penreco). Gels comprising mineral oil, hydrogenated polyisobutene, isoparaffins, such as isohexadecane or isododecane, and also comprising ester oils, in particular comprising isopropylpalmitate or isopropylmyristate, are preferred.

Compositions that are preferred in accordance with the invention are characterized in that they include at least one ethylene/propylene/styrene copolymer and/or butylene/ethylene/styrene copolymer in a total amount of 0.05-3% by weight, preferably 0.1-2% by weight, particularly preferably 0.2-1.0% by weight, extremely preferably 0.3-0.5% by weight, in each case in relation to the total weight of the composition according to the invention.

Further lipophilic thickeners that are preferred in accordance with the invention are selected from silicone elastomers. A further preferred embodiment of the invention is characterized in that at least one silicone elastomer, which is obtainable by the crosslinking of an organopolysiloxane, which includes at least 2 C₂-C₁-alkenyl groups with terminal double bond in each molecule, with an organopolysiloxane that has at least 2 silicone-bound hydrogen atoms in each molecule, is included.

Organopolysiloxanes having at least 2 C₂-C₁₀ alkenyl groups with terminal double bond in the molecule that are particularly preferred in accordance with the invention are selected from methylvinylsiloxanes, methyl vinyl siloxane-dimethylsiloxane copolymers, dimethylpolysiloxanes with dimethylvinylsiloxy terminal groups, dimethylsiloxane-methylphenylsiloxane copolymers with dimethylvinylsiloxy terminal groups, dimethyl siloxane-diphenylsiloxane-methylvinylsiloxane copolymers with dimethylvinylsiloxy terminal groups, dimethylsiloxane-methylvinylsiloxane copolymers with trimethylsiloxy terminal groups, dimethylsiloxane-methylphenylsiloxane-methylvinylsiloxane copolymers with trimethylsiloxy end groups, methyl (3,3,3-trifluoropropyl) polysiloxanes with dimethylvinylsiloxy terminal groups and dimethylsiloxane-methyl (3,3,3-trifluoropropyl) siloxane copolymers with dimethylvinylsiloxy terminal groups.

Crosslinking organopolysiloxanes having at least two silicone-bound hydrogen atoms that are particularly preferred in accordance with the invention are selected from methyl hydrogen polysiloxanes with trimethylsiloxy terminal groups, dimethylsiloxane-methyl hydrogen siloxane copolymers trimethylsiloxy terminal groups, and cyclic dimethylsiloxane-methyl hydrogen siloxane copolymers.

Silicone elastomers which are particularly preferred in accordance with the invention and which as raw material are already present pre-swollen in a silicone that is liquid at room temperature under normal conditions and constitute a silicone-based gel are commercially attainable, for example under the trade names Dow Corning 9040 Silicone Elastomer Blend (a cyclodimethicone (and) dimethicone crosspolymer from Dow Corning; silicone elastomer content 12-13% by weight), SFE 168, a cyclomethicone (and) dimethicone/vinyl dimethicone crosspolymer from GE Silicones, vinyl dimethicone crosspolymers, included in KSG-15 (cyclomethicone (and) dimethicone/vinyl dimethicone crosspolymer, silicone elastomer content 4-10% by weight), KSG-16 (dimethicone (and) dimethicone/vinyl dimethicone crosspolymer, silicone elastomer content 20-30% by weight), KSG-17 (cyclomethicone (and) dimethicone/vinyl dimethicone crosspolymer), KSG-18 (phenyl trimethicone (and) dimethicone/phenyl vinyl dimethicone crosspolymer, silicone elastomer content 10-20% by weight_; and KSG-20, obtainable from Shin Etsu Silicones f America (Akron, Ohio), and from Grant Industries Inc. (Elmwood Park, N.J.) the products from the Gransil® range, in particular Gransil SR-CYC (cyclomethicone and stearyl-vinyl/hydromethylsiloxane copolymer), Gransil® RPS Gel (INCI name: Cyclopentasiloxane and Polysilicone-11), Gransil® GCM-4 (INCI name: Cyclotetrasiloxane and Polysilicone-11), Gransil®GCM-5 (INCI name: Cyclopentasiloxane and Polysilicone-11), Gransil® RPS (INCI name: Cyclopentasiloxane and Polysilicone-11), GI-CD 10 (INCI name: Cyclopentasiloxane (and) Stearoxymethicone/Dimethicone Copolymer (and) Dimethicone), Gransil® IDS (INCI name: Isododecane (and) Cyclotetrasiloxane (and) Polysilicone-11), Gransil®PC-12 (INCI name: Isododecane (and) Polysilicone-11), Gransil® IDS-5 (INCI name: Isododecane (and) Cyclopentasiloxane (and) Polysilicone-11), Gransil® APK-1 (INCI name: Dimethicone and Cyclopentasiloxane and Polysilicone-11 and Nylon-12 and Methyl Methacrylate/Acrylonitrile Copolymer and PEG-10 Dimethicone and Polysorbate-40 and Isohexadecane and Ammonium Polyacryloyldimethyl Taurate), Gransil® DMCM-5 (INCI name: Dimethicone and Cyclopentasiloxane and Polysilicone-11), Gransil® DMG-6 with dimethicone (6 cSt) (INCI name: Dimethicone and Polysilicone-11), Gransil® DMG-20 with dimethicone (20 cSt) (INCI name: Dimethicone and Polysilicone-11), Gransil®AM-8 Gel (INCI name: Caprylyl Methicone and Cyclopentasiloxane and Polysilicone-11), Gransil®DM 5 with dimethicone (5 cSt) (INCI name: Dimethicone and Polysilicone-11), Gransil® DMID (INCI name: Dimethicone and Isododecane and Polysilicone-11), Gransil® PM (INCI name: Phenyl Trimethicone and Polysilicone-11), Gransil® ININ (INCI name: Isononyl Isononanoate (and) Polysilicone-11).

Silicone elastomers which as raw material are already present pre-swollen, mixed with a non-silicone-containing oil, fat or wax, in a silicone that is liquid at room temperature under normal conditions, and which represent a silicone/non-silicone-based gel also can be used with preference in the compositions according to the invention. Silicone elastomer compositions of this type are also commercially available, for example under the trade names Gransil® MLB (INCI name: Cyclopentasiloxane and Polysilicone-11 and Beeswax), Gransil® PS (INCI name: Cyclotetrasiloxane and Polysilicone-11 and Petrolatum), Gransil® PS-5 (INCI name: Cyclopentasiloxane and Polysilicone-11 and Petrolatum), Gransil® DMG-20 P with dimethicone (20 cSt) and Petrolatum (INCI name: Dimethicone and Polysilicone-11 and Petrolatum), Gransil® RJO (INCI name: Cyclopentasiloxane and Polysilicone-11 and Jojoba Oil), Gransil® LANO (INCI name: Cyclopentasiloxane and Polysilicone-11 and Lanolin), Gransil® OHS-5 (INCI name: Cyclopentasiloxane and Polysilicone-11 and Octyl Hydroxystearate) and Gransil® DML (INCI name: Dimethicone (and) Neopentyl Glycol Diheptanoate (and) Polysilicone-11).

A further preferred embodiment of the invention is characterized in that the silicone elastomer is obtainable by the cross-linking of an organopolysiloxane, which includes at least 2 C₂-C₁₀ alkenyl groups with terminal double bond in each molecule, with at least one alpha, omega diene. Cross-linking alpha, omega dienes that are particularly preferred in accordance with the invention have the formula CH₂═CH(CH₂)ₓCH═CH₂ with x=1-20. Particularly preferred alpha, omega dienes are selected from 1,4-pentadiene, 1,5-hexadiene, 1,6-heptadiene, 1,7-octadiene, 1,8-nonadiene, 1,11-dodecadiene, 1,13-tetradecadiene, and 1,19-eicosadiene.

Compositions that are preferred in accordance with the invention are characterized in that they include at least one silicone elastomer in a total amount of 0.05-3% by weight, preferably 0.1-2% by weight, particularly preferably 0.2-1.0% by weight, extremely preferably 0.3-0.5% by weight, in each case in relation to the total weight of the composition according to the invention.

As a wax that is solid under normal conditions, the agent according to the invention preferably additionally includes at least one polyethylene wax. Preferred suitable polyethylene waxes have 30 to 60 carbon atoms.

Further preferred compositions according to the invention are characterized in that they include at least one fragrance and/or at least one perfume oil.

Individual aromatic compounds can be used as fragrances or perfume oils, for example the synthetic products of the type of the esters, ethers, aldehydes, ketones, alcohols and hydrocarbons. Phenolic aromatic compounds include, for example, Carvacrol. Aromatic compounds of the type of the esters are, for example, benzyl acetate, methyl anthranilate, ortho-t-butylcyclohexyl acetate, p-tert-butylcyclohexyl acetate, diethyl phthalate, nonanediol-1,3-diacetate, iso-nonyl acetate, iso-nonyl formate, phenylethyl phenylacetate, phenoxyethyl isobutyrate, linalyl acetate, dimethyl benzyl carbinyl acetate, phenylethyl acetate, linalyl benzoate, benzyl formate, ethyl methyl phenyl glycinate, allyl cyclohexyl propionate, styrallyl propionate, benzyl salicylate, ethyl salicylate, iso-amyl salicylate, hexyl salicylate and 4-nonanolide. The ethers include, for example, benzyl ethyl ether, the aldehydes include, for example, the linear alkanals having 8 to 18 C atoms, citral, citronellal, citronellyloxy acetaldehyde, cyclamenaldehyde, hydroxycitronellal, lilial and bourgeonal, the ketones include, for example, 6-acetyl-1,1,3,4,4,6-hexamethyl tetrahydronaphthalene, para-t-amylcyclohexanone, 2-n-heptylcyclopentanone, O-methyl naphthyl ketone and the ionones α-isomethylionone and methyl cedryl ketone, the alcohols include cinnamic alcohol, anethole, citronellol, dimyrcetol, eugenol, geraniol, linalool, phenyl ethyl alcohol, and terpineol, the hydrocarbons include 1,3,4,6,7,8-hexahydro-4,6,6,7,8,8-hexamethylcyclopenta-a-2-benzopyran, hydroxymethyl isopropylcyclopentane, 3-a-methyldodecahydro-6,6,9a-trimethylnaphtho-2(2,1-b) furan, iso-butylquinoline and the terpenes and balsams. Preference is given to mixtures of different aromatic compounds which together produce a pleasing fragrance note.

Suitable perfume oils can also include natural aromatic compound mixtures, as are accessible from plant or animal sources, for example pine, citrus, jasmine, rose, lily, or ylang-ylang oil. Less volatile essential oils, which are usually used as aroma components, are also suitable as perfume oils, for example sage oil, chamomile oil, melissa oil, mint oil, cinnamon leaf oil, lime blossom oil, juniper berry oil, vetivert oil, olibanum oil, galbanum oil, laudanum oil, clove oil, isoeugenol, thyme oil, bergamot oil, geranium oil, and rose oil.

Preferred compositions according to the invention include at least one fragrance and/or at least one perfume oil in a total amount of 0.01-10% by weight, preferably 0.1-5% by weight, particularly preferably 0.5-3% by weight, extremely preferably 1-2% by weight, in each case in relation to the total weight of the composition according to the invention.

Preferred compositions according to the invention are characterized in that they additionally include at least one deodorant active ingredient. Preferred deodorant active ingredients of this type are selected from odor absorbers, ion exchangers that act in a deodorizing manner, anti-bacterial substances, prebiotically active substances, and enzyme inhibitors and, particularly preferably, combinations of the aforementioned deodorant active substances.

Silicates serve as odor absorbers which also at the same time advantageously support the rheological properties of the composition according to the invention. Silicates that are particularly preferred in accordance with the invention include, in particular, sheet silicates and, among these, in particular montmorillonite, kaolinite, illite, beidellite, nontronite, saponite, hectorite, bentonite, smectite, and talc. Further advantageous odor absorbers are, for example, zeolites, zinc ricinoleate, cyclodextrins, certain metal oxides, such as alumina, and chlorophyll.

Anti-bacterial or anti-microbial active ingredients are understood in accordance with the invention to mean active ingredients which reduce the number of skin bacteria involved in the development of odor and/or inhibit the growth of said bacteria. These bacteria include, inter alia, various species from the group of staphylococci, the group of Coryne bacteria, anaerococci and micrococci.

In particular, organohalogen compounds and halides, quaternary ammonium compounds, a number of plant extracts and zinc compounds are preferred in accordance with the invention as anti-bacterial or anti-microbial active substances. These include, inter alia, triclosan, chlorohexidine and chlorohexidine gluconate, 3,4,4′-trichlorocarbanilide, bromchlorophene, dichlorophene, chlorothymol, chloroxylenol, hexachlorophene, dichloro-m-xylenol, dequalinium chloride, domiphene bromide, ammonium phenolsulfonate, benzalkonium halides, benzalkonium cetyl phosphate, benzalkonium saccharinates, benzethonium chloride, cetyl pyridinium chloride, lauryl pyridinium chloride, lauryl isoquinolinium bromide, and methylbenzedonium chloride. Furthermore, phenol, disodium dihydroxy ethyl sulfosuccinyl undecylenate, sodium bicarbonate, zinc lactate, sodium phenolsulfonate and zinc phenolsulfonate, ketoglutaric acid, terpene alcohols such as farnesol, chlorophyllin-copper complexes, α-monoalkyl glycerol ethers having a branched or linear, saturated or unsaturated, optionally hydroxylated C₆-C₂₂ alkyl group, particularly preferably α-(2-ethylhexyl) glycerol ether, commercially available as Sensiva® SC 50 (ex Schülke & Mayr), carboxylic acid esters of mono-, di- and triglycerol (for example glycerol monolaurate, diglycerol monocaprinate), lantibiotics, and plant extracts (for example green tea and constituents of lime blossom oil) can be used.

Further preferred deodorant active ingredients are selected from what are known as prebiotically active components, which are to be understood in accordance with the invention to include components which only inhibit, or which at least primarily inhibit the odor-forming bacteria of the skin microflora, but not the desired bacteria, i.e. the non-odor-forming bacteria, which belong to a healthy skin microflora. The active ingredients which are disclosed as being prebiotically active in laid-open application documents DE 10333245 and DE 102004100968 are explicitly included here; these include conifer extracts, in particular from the group of the Pinaceae, and plant extracts from the group of Sapindaceae, Araliaceae, Lamiaceae and Saxifragaceae, in particular extracts from Picea spp., Paullinia sp., Panax sp., Lamium album or Ribes nigrum and mixtures of these substances. The enzyme inhibitors include substances that are responsible for sweat decomposition enzymes, in particular which inhibit arylsulfatases, β-glucuronidases, aminoacylases, esterases, lipases and/or lipoxygenases, for example trialkyl citric acid esters, in particular, triethyl citrate, or zinc glycinate. Preferred compositions according to the invention are characterized in that the at least selected one additional deodorant active ingredient is selected from arylsulfatase inhibitors, β-glucuronidase inhibitors, aminoacylase inhibitors, esterase inhibitors, lipase inhibitors and lipoxygenase inhibitors, α-monoalkyl glycerol ethers with a branched or linear saturated or unsaturated, optionally hydroxylated C₆-C₂₂ alkyl group, in particular α-(2-ethylhexyl) glycerol ether, prebiotically active components, trialkyl citric acid esters, in particular, triethyl citrate, active ingredients which reduce the number of the skin bacteria involved in odor development from the group of staphylococci, Coryne bacteria, anaerococci and micrococci or inhibit the growth thereof, zinc compounds, especially zinc phenolsulfonate and zinc ricinoleate, organohalogen compounds, in particular triclosan, chlorhexidine, chlorhexidine gluconate and benzalkonium halides, quaternary ammonium compounds, in particular cetylpyridinium chloride, odor absorbers, in particular silicates and zeolites, sodium bicarbonate, lantibiotics, and mixtures of the aforementioned substances.

Further preferred compositions according to the invention are characterized in that the at least one additional deodorant active ingredient is included in a total amount of 0.1-10% by weight, preferably 0.2-7% by weight, particularly preferably 0.3-5% by weight, in relation to the total weight of the active substance in the total composition.

Antioxidant substances can counteract the oxidative decomposition of the sweat components and in this way can inhibit odor development. Suitable antioxidants are imidazole and imidazole derivatives (for example urocanic acid), peptides such as D,L-carnosine, D-carnosine, L-carnosine and derivatives thereof (for example anserine), carotenoids, carotenes (for example α-carotene, β-carotene, lycopene) and derivatives thereof, lipoic acid and derivatives thereof (for example dihydrolipoic acid), aurothioglucose, propylthiouracil and other thio compounds (for example thioglycerol, thiosorbitol, thioglycolic acid, thioredoxin, glutathione, cysteine, cystine, cystamine and their glycosyl, N-acetyl, methyl, ethyl, propyl, amyl, butyl, lauryl, palmitoyl, oleyl, γ-linoleyl, cholesteryl and glyceryl esters) and their salts, dilauryl thiodipropionate, distearyl, thiodipropionate, thiodipropionic acid and derivatives thereof (esters, ethers, peptides, lipids, nucleotides, nucleosides and salts) and sulfoximine compounds (for example buthionine sulfoximines, homocysteine sulfoximine, buthionine sulfone, penta-, hexa-, and heptathionine sulfoximine) in very low tolerated doses (for example pmol/kg to μmol/kg), and also metal chelators (for example α-hydroxy fatty acids, EDTA, EGTA, lactoferrin), humic acids, bile acid, bile extracts, catechins, bilirubin, biliverdin and derivatives thereof, unsaturated fatty acids and their derivatives (for example γ-linolenic acid, linoleic acid, arachidonic acid, oleic acid), folic acid and derivatives thereof, hydroquinone and its derivatives (for example arbutin), ubiquinone and ubiquinol and derivatives thereof, vitamin C and derivatives thereof (for example ascorbyl palmitate, stearate, dipalmitate, acetate, Mg ascorbyl phosphates, sodium and magnesium ascorbate, disodium ascorbyl phosphate and sulfate, potassium ascorbyl tocopheryl phosphate, chitosan ascorbate), isoascorbic acid and derivatives thereof, tocopherols and their derivatives (for example tocopheryl acetate, linoleate, oleate and succinate, Tocophereth-5, Tocophereth-10, Tocophereth-12, Tocophereth-18, Tocophereth-50, Tocophersolan), vitamin A and derivatives (for example vitamin A palmitate), the coniferyl benzoate of benzoin resin, rutin, rutinic acid and derivatives thereof, dosodiumrutinyl disulfate, cinnamic acid and derivatives thereof, kojic acid, chitosan glycolate and salicylate, butylhydroxytoluene, butylhydroxyanisole, nordihydroguajak resin acid, nordihydroguaiaretic acid, trihydroxybutyrophenone, uric acid and derivatives thereof, mannose and derivatives thereof, selenium and selenium derivatives (for example selenomethionine), stilbenes and stilbene derivatives (for example stilbene oxide, trans-stilbene oxide). Suitable derivatives (salts, esters, sugars, nucleotides, nucleosides, peptides and lipids) and mixtures of these mentioned active ingredients or plant extracts including these antioxidants can be used in accordance with the invention.

Tocopherol and derivatives thereof, in particular tocopheryl acetate, and carotinoids and butylhydroxytoluene/anisol are preferred as lipophilic, oil-soluble antioxidants from this group.

The total amount of antioxidants in preferred preparations according to the invention is 0.001-10% by weight, preferably 0.05-5% by weight, and in particular 0.1-2% by weight, in relation to the total preparation.

Complexing agent substances can also support the deodorizing effect in that they complex the oxidatively acting heavy metal ions (for example iron or copper) in a stable manner. Suitable complexing agents are selected from the above-mentioned complexing agents.

A second subject of the invention is the use of mixtures of
a) at least one alkoxy-modified silicone, as defined in the description of the first subject of the invention, and
b) at least one ester of a C₈-C₁₄ alkanoic acid and a C₈-C₁₄ alkanol, as defined in any one of the preceding claims, for
   increasing the antiperspirant effect of antiperspirant active ingredients (C) from the group of aluminum salts or aluminum-zirconium salts.

A third subject of the invention is the use of mixtures of
a) at least one alkoxy-modified silicone, as defined in the description of the first subject of the invention, and
b) at least one ester of a C₈-C₁₄ alkanoic acid and a C₈-C₁₄ alkanol, as defined in any one of the preceding claims, for
   improving the texture of antiperspirant sticks which include at least one antiperspirant active ingredient (C) from the group of aluminum salts or aluminum-zirconium salts.

That which has been said with regard to the agents according to the invention (i.e. the antiperspirant sticks) applies, mutatis mutandis, with regard to preferred embodiments of the use according to the invention.

### EXAMPLES

### 1. Series of Tests with Regard to Texture and Sweat Reduction

The following formulations were produced (all values are in % by weight (active sub stance))

Antiperspirant sticks

| | V1 | V2 | E |
|---|---|---|---|
| Aluminum zirconium trichlorohydrex gly | 17.8 | 17.8 | 17.8 |
| (AAZG 531 Summit Reheis) (content of | (13.97) | (13.97) | (13.97) |
| active substance = 78.5%) | | | |
| Stearyl alcohol | 19.5 | 19.5 | 19.5 |
| Cetyl alcohol | 0.1 | 0.1 | 0.1 |
| PPG-14 butyl ether (Ucon Fluid AP, Dow) | 6.0 | 6.0 | 6.0 |
| Talc | 3.0 | 3.0 | 3.0 |
| Hydrogenated castor oil (Cutina HR, BASF) | 2.8 | 2.8 | 2.8 |
| Propylene glycol dibenzoate (Lexfeel | 2.0 | 2.0 | 2.0 |
| Shine, Inolex) | | | |
| Myristyl myristate (tetradecyl | — | 1.9 | 1.9 |
| tetradecanoate) | | | |
| PEG-12 dimethicone (Xiameter OFX 0193 | 4.0 | — | 4.0 |
| fluid) | | | |
| Cyclopentasiloxane (Xiameter 0245) | to 100 | to 100 | to 100 |
| Sweat reduction (%) | — | 36 | 45 |
| Product delivery to the skin | too little | too little | good |
| Feel on the skin | rough | creamy | creamy |

The formulations were tested with regard to their antiperspirant efficacy in a sauna test. For this purpose, the products were applied to the backs of 25 test individuals in defined areas on 4 successive days. A control area opposite the test field in question remained untreated.

Twenty-four hours after the last product application, the development of sweat was triggered in a sauna at 80° C. for 15 minutes. Absorbent pads were applied beforehand to the areas and were weighed at the end of the sweating phase in order to determine the amount of sweat.

The table shows that with the formulation E according to the invention it was possible to achieve a significant improvement of the sweat reduction compared to the control V2. The formulation V1 was not examined in respect of the sweat reduction; here, the product delivery to the skin was much too low and the feel on the skin was too rough.

Only in the case of a combination of myristyl myristate with PEG-12 dimethicone and the antiperspirant active ingredient (formulation E) were all desired application properties attained.

The stick with the formulation E retained a creamy texture, even after repeated use, with optimal product delivery to the skin.

### 2. Formulation Examples

Antiperspirant stick

| | Example | | | | |
|---|---|---|---|---|---|
| | 1 | 2 | 3 | 4 | 5 |
| Aluminum zirconium trichlorohydrex gly | 17.8 | 17.8 | 20.0 | — | — |
| (AAZG 531 Summit Reheis) | | | | | |
| Aluminum chlorohydrate (Microdry 3115, | — | — | — | 17.8 | 17.8 |
| Summit Reheis) | | | | | |
| Stearyl alcohol | 19.5 | 19.5 | 19.5 | 19.5 | 19.5 |
| Cetyl alcohol | 0.4 | 0.4 | 0.4 | 0.4 | 0.4 |
| Arachidyl alcohol | 0.1 | 0.1 | 0.1 | 0.1 | 0.1 |
| PPG-14 butyl ether (Ucon Fluid AP, Dow) | 6.0 | 6.0 | 6.0 | 6.0 | 6.0 |
| Talc | 3.0 | 3.0 | 3.0 | 3.0 | 3.0 |
| Hydrogenated castor oil (Cutina HR, BASF) | 2.8 | 2.8 | 2.8 | 2.8 | 2.8 |
| Propylene glycol dibenzoate (Lexfeel Shine, | 2.0 | 2.0 | 2.0 | 2.0 | 2.0 |
| Inolex) | | | | | |
| Myristyl myristate (Crodamol MM, Croda) | 1.9 | 2.5 | 1.5 | 1.9 | 1.0 |
| PEG-12 dimethicone (Xiameter OFX 0193 | 4.0 | 3.0 | 6.0 | 4.0 | 1.0 |
| fluid) | | | | | |
| Cyclopentasiloxane (Xiameter 0245) | to 100 | to 100 | to 100 | to 100 | to 100 |

Antiperspirant stick (soft solid)

| | Example | | | |
|---|---|---|---|---|
| | 6 | 7 | 8 | 9 |
| C20-C40 alcohols (Performacol 425, Baker | 2.7 | 2.9 | 5.0 | 2.7 |
| Petrolite) | | | | |
| Dow Corning 9040 Silicon Elastomer Blend | 0.6 | 5.0 | 3.0 | 0.6 |
| Stearyl alcohol | 3.8 | 2.1 | 3.8 | 3.8 |
| Talc | 2.0 | — | — | 2.0 |
| Aluminum Starch Octenylsuccinate (Dry Flo PC, | 8.0 | — | — | 5.0 |
| National Starch) | | | | |
| Sorbitol/Sebacic Acid Copolymer Behenate | 1.8 | — | — | 3.0 |
| (Syncrowax ORM-PW, Croda) | | | | |
| Silica (Aerosil 300, Evonik) | 0.5 | 0.5 | 0.5 | — |
| Pentaerythrityl Distearate (Cutina PES, BASF) | 1.0 | 1.0 | 1.0 | — |
| Aluminum zirconium trichlorohydrex gly (AAZG | 20.0 | 20.0 | 20.0 | 20.0 |
| 531, Summit Reheis) | | | | |
| Myristyl myristate (Crodamol MM, Croda) | 1.9 | 2.5 | 1.0 | 2.5 |
| PEG-12 dimethicone (Xiameter OFX 0193 fluid) | 4.0 | 6.0 | 1.0 | 3.0 |
| Cyclopentasiloxane (Xiameter 0245) | to 100 | to 100 | to 100 | to 100 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant stick, comprising:
a) at least one alkoxy-modified silicone,
b) at least one ester of a C8-C14 alkanoic acid and a C8-C14 alkanol, and
c) at least one antiperspirant active ingredient from the group consisting of aluminum salts or aluminum-zirconium salts.

2. The antiperspirant stick according to claim 1, wherein the antiperspirant stick includes an alkoxy-modified silicone (A) has at least one structural unit of formula (A), 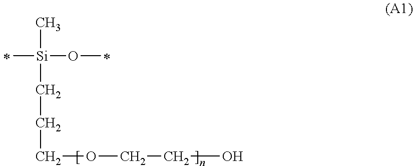
in which n stands for an integer from 2 to 20.

3. The antiperspirant stick according to claim 1, wherein it includes an alkoxy-modified silicone (A) which is composed of structural units of formula (A1), formula (A2), formula (A3), and formula (A3′), 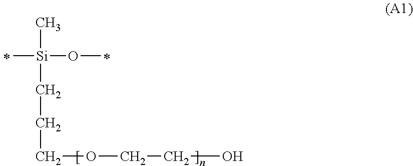
in which n (independently in each structural unit (A1)) stands in each case for an integer from 2 to 20,

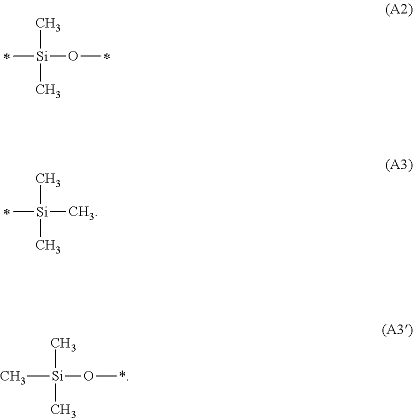

4. The antiperspirant stick according to claim 1, wherein the stick includes at least one alkoxy-modified silicone (A) having a molar mass of from 800 to 10,000 g/mol.

5. The antiperspirant stick according to claim 1, wherein the stick includes at least one alkoxy modified silicone (A) having a molar mass of from 2,500 to 5,000 g/mol.

6. The antiperspirant stick according to claim 1, wherein the stick includes an ester (B) of formula (B1), 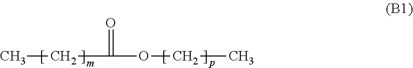
in which m stands for an integer from 6 to 12 and p stands for an integer from 7 to 13.

7. The antiperspirant stick according to claim 1, wherein the stick includes an ester (B) of formula (B1), 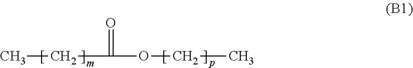
in which m stands for an integer from 6 to 12 and p stands for an integer from 7 to 13.

8. The antiperspirant stick according to claim 5, wherein the stick includes an ester (B) of formula (B1) in which the numbers m and p satisfy the following equation:
m+1=p.

9. The antiperspirant stick according to claim 1, wherein the stick has a water content of at most 10.0% by weight in relation to the total weight of the stick.

10. The antiperspirant stick according to claim 1, wherein the stick has a water content of at most 1% by weight in relation to the total weight of the stick.

11. The antiperspirant stick according to claim 1, wherein the stick includes one or more alkoxy-modified silicones (A) in a total amount of from 0.5 to 10% by weight in relation to the total weight of the stick.

12. The antiperspirant stick according to claim 1, wherein the stick includes one or more alkoxy-modified silicones (A) in a total amount of from 2.1 to 5.2% by weight in relation to the total weight of the stick.

13. The antiperspirant stick according to claim 1, wherein the stick includes one or more esters (B) in a total amount of from 0.5 to 5.0% by weight in relation to the total weight of the stick.

14. The antiperspirant stick according to claim 1, wherein the stick includes one or more esters (B) in a total amount of from 1.6 to 2.5% by weight in relation to the total weight of the stick.

15. The antiperspirant stick according to claim 1, wherein the ratio by weight of all alkoxy-modified silicones (A) included in the stick to all esters (B) included in the stick, (A)/(B), is 1.0 to 4.2.

16. The antiperspirant stick according to claim 1, wherein the ratio by weight of all alkoxy-modified silicones (A) included in the stick to all esters (B) included in the stick, (A)/(B), is 1.8 to 2.

17. The antiperspirant stick according to claim 1, wherein the stick includes one or more antiperspirant active ingredients (C) in a total amount of from 5.0 to 20.0% by weight in relation to the total weight of the stick.

18. The antiperspirant stick according to claim 1, wherein the ratio by weight of all antiperspirant active ingredients (C) included in the stick to the sum of the alkoxy-modified silicones (A) and esters (B) included in the stick, (C)/[(A)+(B)], is 1.0 to 4.5.

19. The antiperspirant stick according to claim 1, wherein the ratio by weight of all antiperspirant active ingredients (C) included in the stick to the sum of the alkoxy-modified silicones (A) and esters (B) included in the stick, (C)/[(A)+(B)], is 1.9 to 2.5.

20. The antiperspirant stick according to claim 1, further comprising one or more polyethylene glycol in a total amount of from 0.01 to 10.0% by weight in relation to the total weight of the stick.
